(19) United States
(12) Patent Application Publication
(10) Pub. No.: US 2013/0117044 A1
(43) Pub. Date: May 9, 2013

(54) SYSTEM AND METHOD FOR GENERATING A MEDICATION INVENTORY

(71) Applicant: James Kalamas, Piedmont, CA (US)

(72) Inventor: James Kalamas, Piedmont, CA (US)

(21) Appl. No.: 13/316,183

(22) Filed: Dec. 9, 2011

Related U.S. Application Data
(60) Provisional application No. 61/556,207, filed on Nov. 5, 2011.

(51) Int. Cl. G06Q 50/24

(52) U.S. Cl. 705/3; 705/2

(57) ABSTRACT

A system and method for electronically verifying a patient's medication inventory comprises receiving an optical image of a medication label on a pill bottle or other medication container, translating said image into text data (e.g., comprising patient's name, medication name, dose, frequency and route of administration of medication); comparing the text data to known medications in a medication database and identifying any identical match. If no match is found, the system and method acts as an expert system to search the data in the medication database for historical user verified closest matches and to return the closest match with the highest user verified historical probability of being correct. The matched information is displayed to a user and the user is enabled to correct the information, if needed. The verified information is stored in a medication database.

### CROSS REFERENCE TO RELATED APPLICATION

This application claims the benefit of U.S. Provisional Application No. 61/556,207 filed Nov. 5, 2011, entitled “SYSTEM AND METHOD FOR GENERATING A MEDICATION INVENTORY” the entirety of which is hereby incorporated by reference.

### BACKGROUND OF THE INVENTION

Medication reconciliation is the process by which a healthcare provider obtains and documents a thorough medication history from a patient. This medication history is an essential first step in any patient encounter with the healthcare system. Failure to correctly construct a complete medication history can delay recognition of adverse drug events, cause under- and over-dosing, cause duplicate therapy, and lead to omissions of therapy. De Winter, et al. demonstrated that 59 percent of patients admitted to the hospital had discrepancies within their medication histories. See De Winter, S., et al., Pharmacist-Versus Physician-acquired Medication History: A Prospective Study at the Emergency Department (Qual Saf Health Care, 2010. 19(5): p. 371-5). This is consistent with many other published studies. According to the Institute of Medicine's Preventing Medication Errors report, the average hospitalized patient is subject to at least one medication error per day. See Preventing Medication Errors 2006, National Academic Press, Washington D.C. (Institute of Medicine). This confirms previous research findings that medication errors represent the most common patient safety error. More than 40 percent of medication errors are believed to result from inadequate medication reconciliation in handoffs of patients during their admission, transfer, and discharge. Of these errors, about 20 percent are believed to result in harm. See Rozich, J. D., et al., Standardization as a Mechanism to Improve Safety in Health Care (Joint Commission J Qual Saf, 2004. 30(1): p. 5-14). As a result, inaccurate collection of medication histories is a leading cause of hospitalization and death in the United States.

The Joint Commission added medication reconciliation across the care continuum as a National Patient Safety Goal in 2005. The Institute for Healthcare Improvement (IHI) has medication reconciliation as part of its 100,000 Lives Campaign. See (http://www.ihi.org/offerings/Initiatives/PastStrategicInitiatives/5MillionLivesCampaign/Documents/Overview%20of%20the%20100K %20Campaign.pdf).

Unfortunately, the process of gathering, organizing, and communicating medication information between a patient and the healthcare system is not straightforward and often relies on the patient to generate their own comprehensive up-to-date medication list. In practice, patients often generate these lists either from memory or by reading the prescription labels on their pill bottles. Several studies have shown that medication lists generated by patients in this way are fraught with inaccuracies, including medication omissions, incomplete dosages, and missing information regarding the administration frequency for each medication. Additionally, the manual transcription of patient-provided medication information into a healthcare provider's medical record system (either paper-based or electronic) is labor intensive, costly, and full of transcription-based errors. Consequently, successful implementation of medication reconciliation has proven difficult and remains challenging.

Therefore, a need exists for a system that can mitigate the errors and inefficiencies inherent in the process of gathering, organizing, and communicating medication information between a patient and the healthcare system that does not rely on a patient's oral medication history at the time of encounter or a patient's ability to manually and accurately provide several fields of data from each of their prescription labels. What is also needed is a system that does not require that healthcare providers be costly scribes to manually transcribe this patient-generated information into the patient's health record.

Several prior art devices are known which use bar code scanners to verify that a correct drug is being administered to a correct patient. See, e.g., Brown, U.S. Pat. No. 4,857,713; Martucci, U.S. Pat. No. 6,985,870; Hochman, U.S. Pat. App. No. 2001/0049608; and Eggers, et al., U.S. Pat. App. No. 2011/00288885. However, all of these prior art devices require medication labels to be in a machine readable format (i.e., a bar code) to enable scanning.

To date, there is no standard machine readable format across all prescription labels; hence some level of manual translation is required to share information between bar code systems. Additionally, none of these prior art devices contemplates the need to capture, verify, and exchange medication inventories, particularly when the patient is interacting with the healthcare system for the first time. Instead, the focus of these devices is to ensure that, at the time a drug is being administered to the patient, it is in fact the drug the patient's caregiver intended be administered.

Jenkins, U.S. Pat. No. 6,597,392, discloses a device and method for capturing various medical images that are then transmitted to a remote computer. This disclosure teaches the photographing of prescription drug label images. These photos are then stored and available for later viewing by a physician or other party. These images are also retained in a patient's medical record but only as an image, not as discrete data elements. However, for the information to be electronically cross-referenced (or reconciled) with a medication library and/or imported into an electronic medical record (EMR) in a reportable fashion, the user of the system would need to manually transcribe the information in the captured image into discrete data elements. Thus, the method and system taught by Jenkins does not obviate the problems inherent in manually transcribing discrete data elements (e.g., names of medications, dosage, frequency of administration) into an electronic form.

Spero, et al., U.S. Pat. No. 7,069,240, discloses a system and method for capturing and storing expense receipts. This disclosure teaches the capture of expense receipt images from which information can be extracted and stored into an expense reporting form. However, this patent does not teach the use of a system, e.g., an expert system supported by machine learning, to automatically reconcile optically captured information with a known database of relevant information. In the field of medication reconciliation, which is not contemplated by Spero, et al., this functionality is essential to automate the creation of accurate medication inventories, thereby diminishing the harmful and expensive human errors that are inherent in all medication reconciliation approaches that exist to date.

Consequently, there is a need for a computer system and method that captures human readable information on any prescription label (e.g., pill bottle, pill box, prescription bag), translates that information into text data, compares the text data with a medication database of established medications, presents the matched medication information for verification by the user (e.g., the patient or healthcare provider), and stores the verified medication in a medication inventory.

### SUMMARY OF THE INVENTION

The system and method according to certain embodiments of the present invention substantially overcome the deficiencies of known systems and methods by generating a medication inventory of the one or more medications a patient is taking from a scan of the human readable information on each of the patient's prescription labels.

In one embodiment of the present invention, a computer implemented method for generating a medication inventory for a user comprises receiving an optical image of a medication label, translating the optical image into computer readable text data, comparing the text data to known medications stored in a first database and identifying any identical match and, if no identical match is found, identifying the closest match, displaying the matched medication to the user, enabling the user to indicate whether the medication is correct, and, where the user indicates the medication is not correct, enabling the user to input the correct medication, and storing the verified medication in a medication inventory database.

In another embodiment of the present invention, a system for generating a medication inventory for a patient comprises an optical scanner for capturing a human readable image of a medication label, a first database for storing a library of known medications, a data processor operative to receive the image, to convert the image into searchable text data, and to compare this text data with the medications in the first database, to identify the medication that most closely matches the text data, and a user interface for displaying to a user the matched medication, the user interface enabling the user to verify that the matched medication is correct and to input the correct medication name if the matched medication is incorrect.

Another embodiment comprises utilizing an optical scanning device for capturing a human readable image; a memory for storing the scanned image; a medication database of all known medications (e.g., prescriptions, vitamins, herbal preparations), their dosages, medication frequencies, and routes of administration; a data processor operative to translate the scanned image into searchable text data and to compare the text data with the data in the medication database; a user interface that allows for the display to a user of the matched prescription label information for user verification; and a communication device for transmission of medication inventory data to other devices.

These and other embodiments, features, aspects, and advantages of the invention will become better understood with reference to the following description, appended claims, and accompanying drawings.

### BRIEF DESCRIPTION OF THE DRAWINGS

FIG. 1 illustrates a block diagram of a system according to one embodiment of the present invention.

FIG. 2 illustrates a block diagram of a system according to an alternative embodiment of the present invention.

FIG. 3 is a flowchart illustrating one embodiment of a method for generating and verifying a patient's medication inventory according to the present invention.

FIG. 4 is a flowchart illustrating one embodiment of a method for finding the closest match for the method illustrated in FIG. 3 according to the present invention.

FIG. 5 shows an exemplary display for presenting the matched medication information generated by the method and system for verification by a user according to an embodiment of the present invention.

FIGS. 6-9 show exemplary displays for enabling a user to provide corrections to the medication information according to an embodiment of the present invention.

Reference symbols or names are used in the Figures to indicate certain components, aspects, or features shown therein, with reference symbols common to more than one Figure indicating like components, aspects or features shown therein.

### DETAILED DESCRIPTION

The system and method according to one embodiment of the present invention includes an optical scanning device, data storage, data analysis, and communication capabilities. The system and method is preferably implemented in a special purpose computer device containing an optical scanner. The computer may be a device including but not limited to a personal computer, computer chip, smartphone, computer tablet device, or the like. The optical scanner can be any type of optical system capable of capturing an image, including but not limited to a camera, digital camera, smartphone with a built-in camera, computer tablet device with a built-in camera, or the like. Alternatively, the system can be implemented as an optical scanning device connected to a server system that is connected to a wide area network accessible from any location connected to the network.

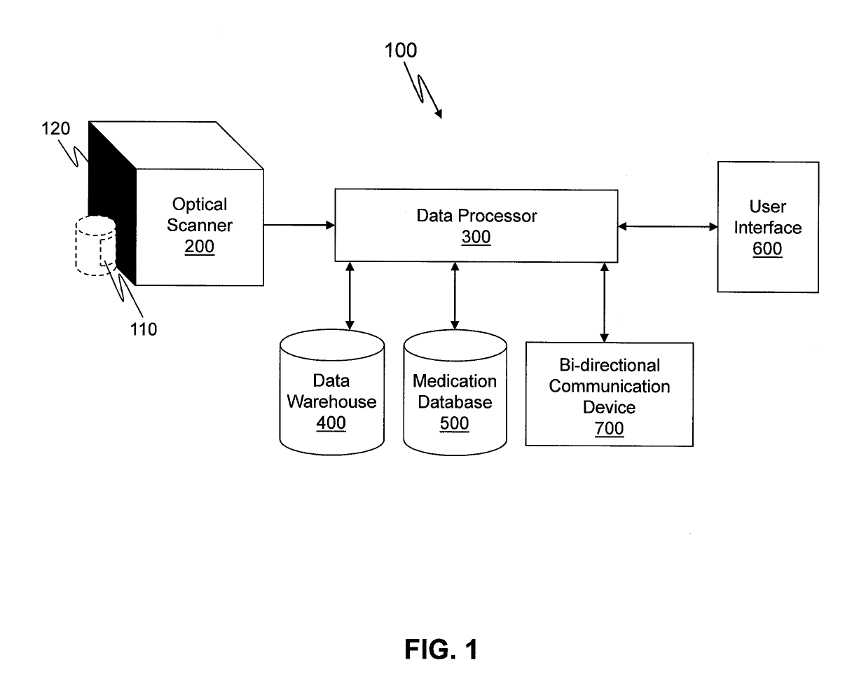

FIG. 1 is a block diagram of a System 100 implemented in accordance with one embodiment of the invention. System 100 includes an Optical Scanner 200, a Data Processor 300, a Data Warehouse 400, a Medication Database 500, and a User Interface 600. A Bi-directional Communication Device 700 may also be included in System 100 to enable remote access to the Medication Database 500 and Data Warehouse 400. Medication Database 500 is also referred to herein as the first database, and Data Warehouse 400 is also referred to herein as the medication inventory database.

Optical Scanner 200 is preferably any optical scanner that can capture a human readable image of a medication label 110, and all the information contained therein, when the label is affixed to a pill bottle. Alternatively, Optical Scanner 200 may be used to capture an image of a medication label affixed to any other surface or a label that is not affixed to any surface. In one embodiment, the image of a medication label generated by Optical Scanner 200 is processed by Data Processor 300 and stored in Data Warehouse 400.

Data Processor 300 is at the core of System 100 and is preferably operates as a computerized expert system having machine learning capabilities. An expert system, broadly defined, is a software system that attempts to reproduce the performance of one or more human experts by analyzing information using what appears to be reasoning capabilities. Machine learning comprises techniques for enabling a computer to learn from either inductive or deductive reasoning by allowing the computer to evolve behaviors based on new data received from sensors and the like.

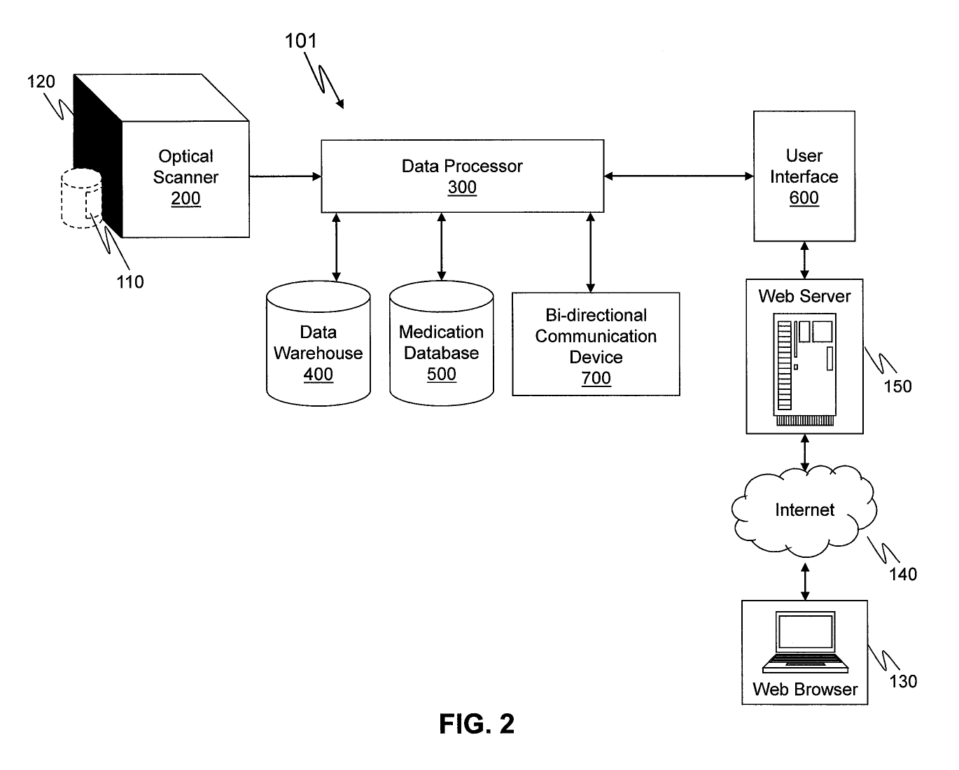

FIG. 2 is a block diagram of an alternative embodiment of a system 101 according to the present invention. In System 101, a user (not shown) is enabled to use any suitable Browser 130 to access the User Interface 600 via the Internet 140 and a Web Server 150.

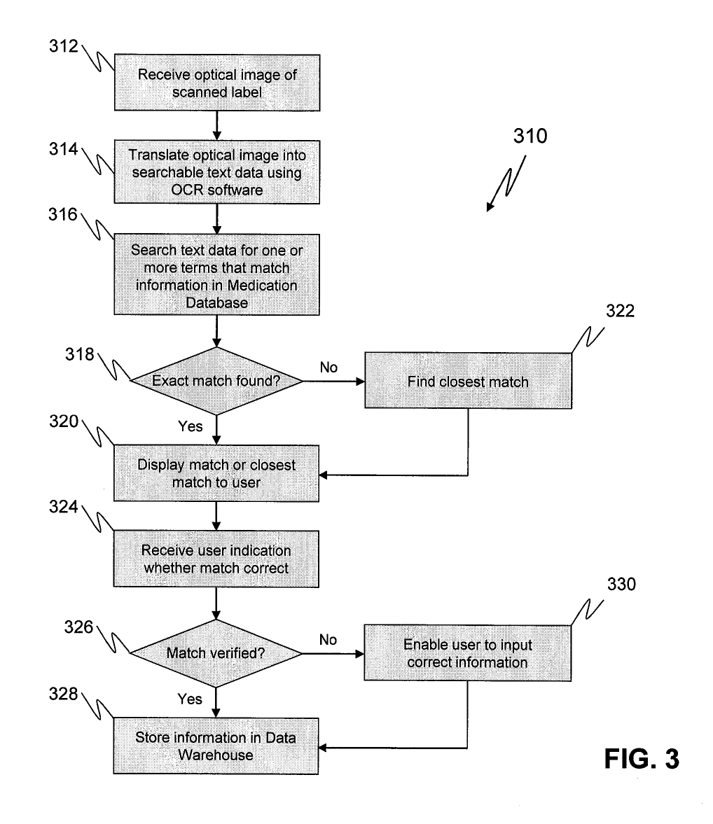

One embodiment of a process for creating a medication inventory is shown in the flowchart at 310 in FIG. 3. This process is initiated at 312 when an electronic image of a scanned label is received. In one embodiment, this is done by a user (not shown) who has placed a medication label 110 facing aperture 120 of Optical Scanner 200, as shown in FIGS. 1 and 2. At 314, the scanned optical image is translated into computer searchable text data. There are a number of ways known in the art for a computer to automatically perform this translation, e.g., with appropriate optical character recognition (OCR) software. At 316, the text data is searched to determine whether one or more fields of data match information in the Medication Database 500. In one embodiment, Data Processor 300 compares the text data to known medications stored in the first database, the Medication Database 500, to identify any identical match. At 318, if an identical (exact) match is found, the match is displayed to a user of the method 310 at 320. If no identical match is found, Data Processor 300 identifies the closest match at 322. The closest match is then displayed to the user at 320.

In one embodiment, at 322, Data Processor 300 searches for the closest match between the text data obtained from the optical image and information in a second database containing stored demographic and medication label data for the patient and historical user verified closest matches for other patients obtained from other users of the system and method. Data Processor 300, acting as an expert system including machine learning capabilities, is operative to search the data in said second database and to identify the closest match having the highest probability of being correct based on said historical user verified closest matches. In one embodiment, the second database is Data Warehouse 400.

At 324, the system and method enables the user to indicate whether the displayed match is correct. At 326, if the user verifies that the match is correct, the verified match is stored in the Data Warehouse 400 at 328. If the user indicates the match is not correct, at 330, the user is enabled to input the correct information. This corrected match is then stored in the Data Warehouse 400 at 328.

According to one embodiment of the invention, the text data translated from the optical image may include one or more of the following categories of information: the patient's name, the name of the medication, the prescribed dosage, the frequency at which the medication should be taken, and the route to be used for administering the medication. The route specified for administering the medication, for example may be orally, if the medication is a pill, the skin if the medication is a cream, a body orifice if the medication is a suppository, or by injection. In this embodiment, Medication Database 500 includes information regarding one or more of the standard dosages, medication frequencies and routes of administration for each of the medications stored in Medication Database 500. Data Processor 300 operates to compare the text data with the information stored in Medication Database 500 to identify the closed match for each category of information included in Database 500. These matches are then each displayed to the user at 320, and the user's indication of whether each match is correct is received. Where the user indicates the medication name, dosage, medication frequency and/or route of administering the medication are correct, the information is stored in Data Warehouse 400 at 328. Where any of the matches are not correct, the user is enabled to input the correct information at 330 and this corrected information is then stored in the Data warehouse 400 at 328.

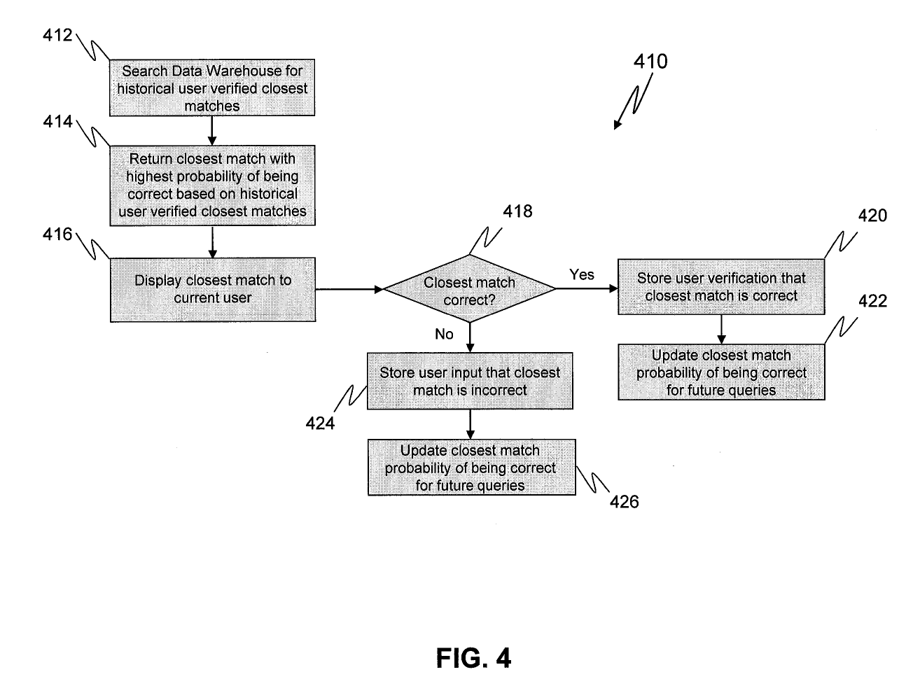

FIG. 4 is a flowchart illustrating one embodiment of a machine learning method 410 for finding the closest match for the method 310 illustrated in FIG. 3 according to the present invention. Method 410 is used when an identical match to the text data translated from the medication label image is not found within Medication Database 500. As seen in FIG. 4, at 412 the Data Warehouse 400 is searched by Data Processor 300 for historical user verified closest matches for each category of medication data stored in the Data Warehouse 400. At 414, the closest match with the highest probability of being correct based on historical user verified closest matches is identified and returned. At 416, this closest match is displayed to the user. As indicated above, this process is repeated for each field of data, i.e., categories of information, stored in Medication Database 500 for each medication. That is, the closest match in each category of medication data is displayed to the user. At 418, the method enables the user to verify whether the closest match in each category is correct. If yes, the user verification of the correctness of the information in each category is stored in the Data Warehouse 400 at 420. At 422, this information is used to update the closest match probability of being correct for use in future queries. If the user indicates that the closest match is incorrect at 418, this information is stored in the Data Warehouse 400 at 424. At 426, this information is used to update the closest match probability of being correct for use in future queries.

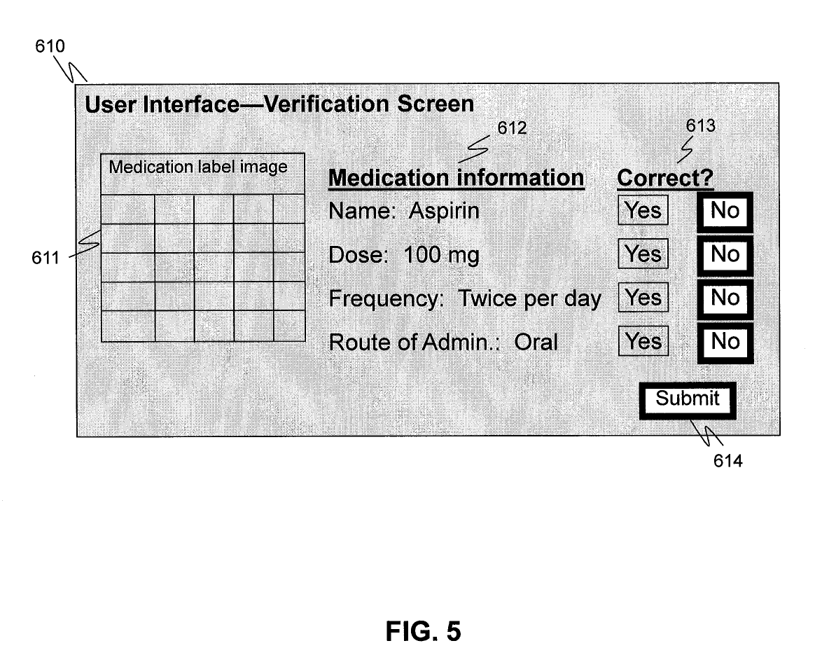

FIGS. 5-9 illustrate exemplary user interface screens employed to elicit the user input described in step 326 of FIG. 3. FIG. 5 illustrates an exemplary User Interface Verification Screen 610. In one embodiment, included in Screen 610 is the medication label image 611 captured by Optical Scanner 200 and stored in Data Warehouse 400. The matched medication information 612 generated by Data Processor 300 is shown next to the label image 611. Next to the medication information 612 is a user verification input 613 and a user Submit button 614. As seen, user verification input 613 includes “Yes” and “No” buttons for each category of medication information 612 shown on Screen 610. User Interface Screen 610 allows the user to compare the matched medication information 612 with the image of the medication label 611 for accuracy. The user indicates whether the matched medication information 612 is correct by selecting “Yes” for each category in user verification input 613 and then selecting “Submit” at 614. As described in FIG. 3 with respect to step 328, this verified matched information is stored in Data Warehouse 400. As seen in the exemplary process shown in FIG. 4, the user verified accuracy of the reconciled medication information is used to improve the accuracy of future reconciled medication information returned by Data Processor 300, thereby enabling machine learning.

Data Processor 300 generates its “best guess” of the medication label text data it is analyzing by comparing the text data for the current patient to historical data preferably stored in Data Warehouse 400 and known medication information in Medication Database 500. The historical data is from other users of the system or from prior uses by the current user, including but not limited to previous guesses by the system and method, accuracy of those guesses, and demographic characteristics of previous users such as age, gender, race, medical condition, or other demographic data that may also be stored in the second database. For example, as shown at 612 of FIG. 5, the system guessed “Aspirin” as the name of the medication on the analyzed medication label image 611. If 100 previous guesses of “Aspirin” by the system yielded 99 user confirmed correct guesses of “Aspirin” and only 1 incorrect guess requiring user correction to “Aspartate,” the system “learns” from this previous data in order to “know” that when it analyzes a medication label image and is deciding between Aspirin and Aspartate, the most likely correct guess is Aspirin, not Aspartate. Therefore, based on incomplete text data translated from the received image of the scanned medication label, the system and method according to one embodiment uses historical data and the process of machine learning to formulate a more accurate guess. In this way, as more users use the system and method, the accuracy of matches will increase and reliance on user corrections will decrease. In addition, other patterns aside from previous guesses and their accuracy, including age, gender, medical condition, and race, may emerge in the historical data acquired using the system and method, and these patterns may be useful in comparison and generation of the best guess.

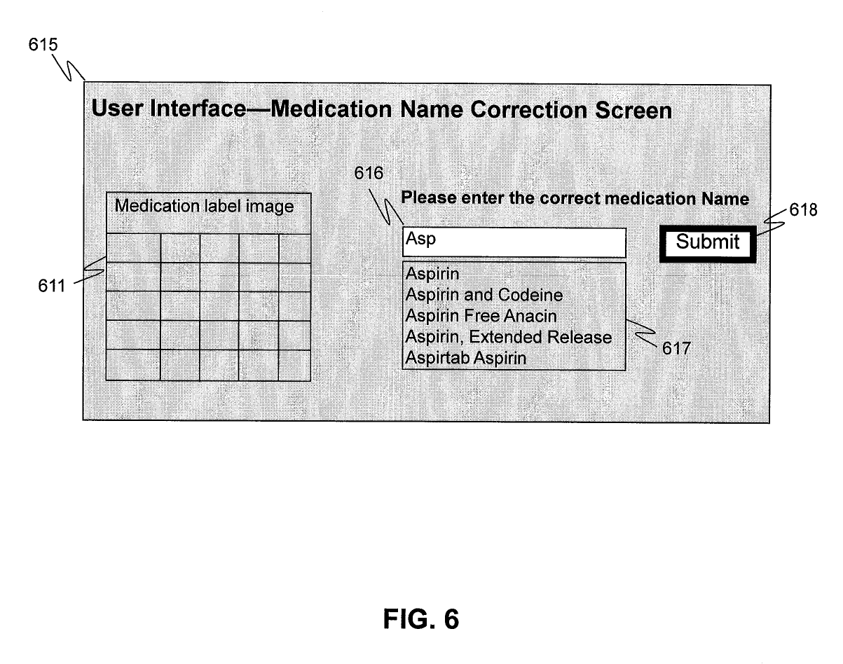

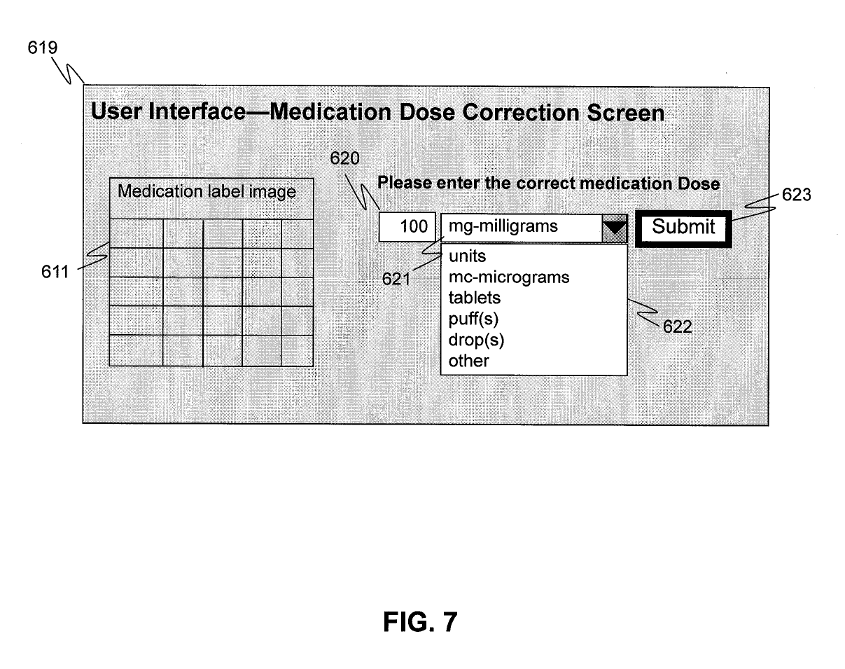

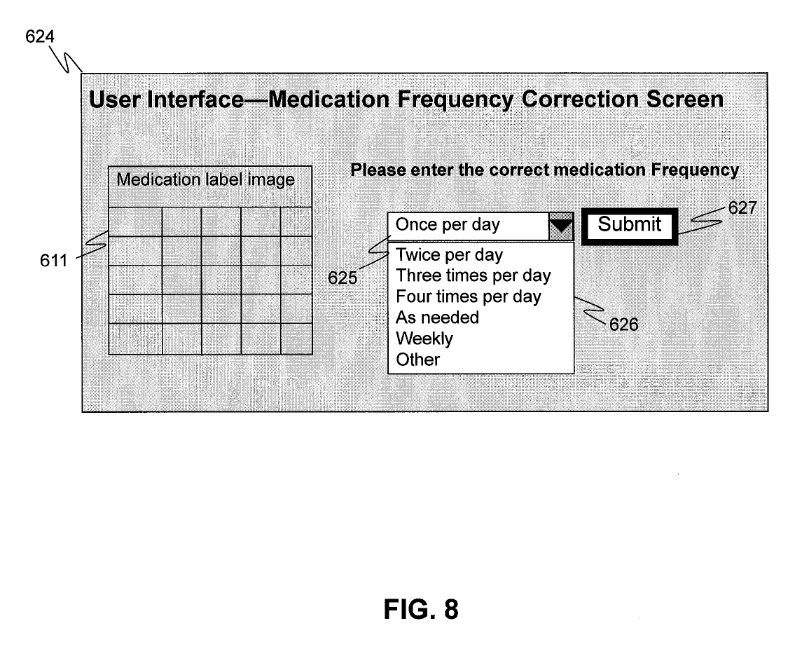

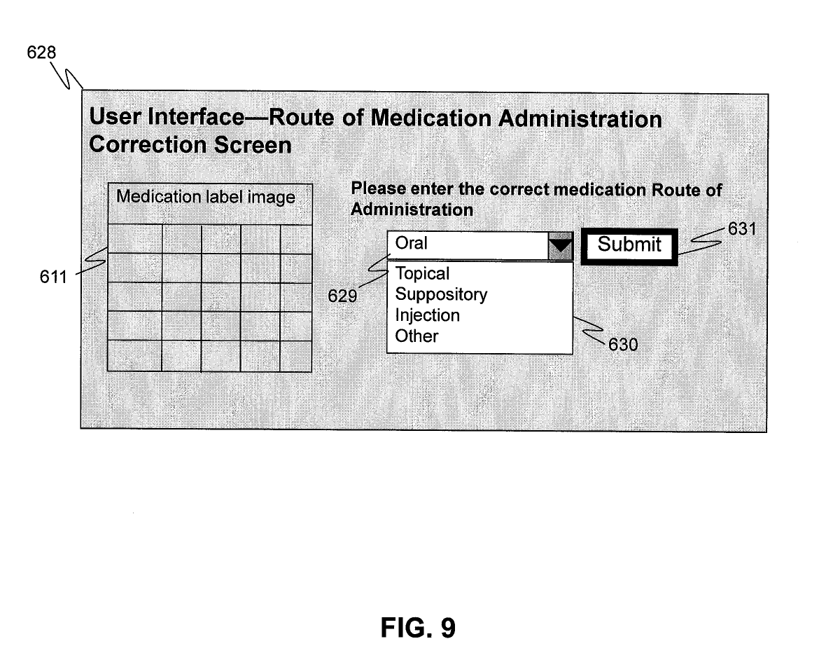

Returning again to exemplary Verification Screen 610, if the user indicates that one or more of the categories of matched medication information 612 is incorrect, the user selects “No”. Once the user selects “Submit” at 614, the user is then presented with successive exemplary User Interface Screens 615 (FIG. 6), 619 (FIG. 7), 624 (FIG. 8), and 628 (FIG. 9), depending on the specific “No” responses to user verification input 613 on User Interface Screen 610. An exemplary embodiment of User Interface Screen 615 for correcting medication name information is illustrated in FIG. 6. User Interface Screen 615 displays medication label image 611 captured by Optical Scanner 200; provides a user input 616 for entry of the user corrected medication name; a medication name drop down menu 617; and a user Submit button 618. User input 616 allows the user to enter the correct medication information (in this example, the name of the medication) as shown on medication label image 611 that is also displayed on User Interface Screen 615 for ease of verification by the user. To facilitate user input of the corrected medication name, a drop down menu 617 is presented to the user and lists known medications containing the first letter or letters of the medication name entered by the user. The user can select from this drop down menu or enter whatever medication name they wish at user input 616. Once corrected, the user selects “Submit” 618 to instruct Data Processor 300 to store the corrected information in Data Warehouse 400 as shown in step 328 in FIG. 3. The fact that Data Processor 300 returned an incorrect match to the information on medication label image 611 is stored in Data Warehouse 400. As before, the user verified accuracy (or inaccuracy in this example) of the matched medication information is used to improve the accuracy of future matched medication information returned by Data Processor 300, thereby enabling machine learning.

The user can enter additional corrected medication information, including, but not limited to dose, frequency, and route of administration by using exemplary User Interface Screens 619, 624, and 628, respectively, in a similar fashion to User Interface Screen 615. User Interface Screen 619 in FIG. 7 presents medication label image 611; provides for a user corrected dose input 620 and a user corrected dose units input 621; a dose drop down list 622; and a user Submit button 623. User Interface Screen 624 in FIG. 8 presents medication label image 611; provides a user corrected frequency input 625; a frequency drop down list 626; and a user Submit button 627. User Interface Screen 628 in FIG. 9 presents medication label image 611; provides a user corrected route of administration input 629; a route of administration drop down list 630; and a user Submit button 631.

The user verified reconciled medication label information stored in Data Warehouse 400 can be subsequently transmitted to any other user or device via Bi-directional Communication Device 700 as illustrated in FIGS. 1 and 2. Bi-directional Communication Device 700 may be any communication device that can transmit and receive information via wireless or physical connection (e.g., Wi-Fi, USB (Universal Serial Bus)).

Having disclosed exemplary embodiments, modifications and variations may be made to the disclosed embodiments while remaining within the scope of the invention as described by the following claims.

The present invention has been described in relation to particular examples, which are intended in all respects to be illustrative rather than restrictive. Those skilled in the art will appreciate that many different combinations of circuits will be suitable for practicing the present invention. Moreover, other implementations of the invention will be apparent to those skilled in the art from consideration of the specification and practice of the invention disclosed herein. It is intended that the specification and examples therein be considered as exemplary only, with a true scope of the invention being indicated by the following claims.

What is claimed is:

1. A computer implemented method for generating a medication inventory for a user, comprising:
receiving an optical image of a medication label;
translating the optical image into computer readable text data;
comparing the text data to known medications stored in a first database and identifying any identical match and, if no identical match is found, identifying the closest match;
displaying the matched medication to the user;
enabling the user to indicate whether the medication is correct;
where the user indicates the medication is not correct, enabling the user to input the correct medication; and
storing the verified medication in a medication inventory database.

2. The method of claim 1, further comprising:
comparing the text data to known dosages for the verified medication stored in said first database and identifying any identical match, and, if no identical match is found, identifying the closest match;
displaying the matched dosage to the user;
enabling the user to indicate whether the dosage is correct; and
where the user indicates the dosage is not correct, enabling the user to input the correct dosage; and
storing the verified dosage in said medication inventory database.

3. The method of claim 2, further comprising:
comparing the text data to known medication frequencies for the verified medication stored in said first database and identifying any identical match, and, if no identical match is found, identifying the closest match;
displaying the matched medication frequency to the user;
enabling the user to indicate whether the medication frequency is correct; and
where the user indicates the medication frequency is not correct, enabling the user to input the correct medication frequency; and
storing the verified medication frequency in said medication inventory database.

4. The method of claim 3, further comprising:
comparing the text data to known medication routes of administration for the verified medication stored in said first database and identifying any identical match, and, if no identical match is found, identifying the closest match;
displaying the matched medication route of administration to the user;
enabling the user to indicate whether the medication route of administration is correct; and
where the user indicates the medication route of administration is not correct, enabling the user to input the correct medication route of administration; and
storing the verified medication route of administration in said medication inventory database.

5. The method of claim 4, wherein the displaying to the user of the matched medication, dosage, medication frequency, and route of administration comprises the display of this data along with the display of the received optical image on a single computer screen accessible to the user.

6. The method of claim 1, wherein the data in said medication inventory database is password protected.

7. The method of claim 1, wherein the data in said medication inventory database is accessible remotely via a computer network.

8. The method of claim 1, wherein the data in said medication inventory database is used to generate an electronic medical record.

9. The method of claim 1, wherein the optical image is translated into computer readable text data using optical character recognition software.

10. The method of claim 1, further comprising identifying a patient's name on the medication label and storing the name in said medication inventory database.

11. The method of claim 1, wherein the displaying of a matched medication to the user further comprises the displaying of the received optical image on a computer screen together with the matched medication.

12. The method of claim 1, further comprising a second database for storing demographic and medication label data for the patient and historical user verified closest matches for other patients obtained using the system; and wherein the identifying of the closest match includes searching the data in said second database and identifying the closest match having the highest probability of being correct based on said historical user verified closest matches.

13. A system for generating a medication inventory for a patient, comprising:
an optical scanner for capturing a human readable image of a medication label;
a first database for storing a library of known medications;
a data processor operative to receive said image, to convert said image into searchable text data, and to compare said text data with the medications in said first database to identify the medication that most closely matches the text data; and
a user interface for displaying to a user the matched medication and for enabling the user to verify that the matched medication is correct and to input the correct medication name if the matched medication is incorrect.

14. The system of claim 13, further comprising:
a second database for storing demographic and medication label data for the patient and historical user verified closest matches for other patients obtained using the system; and wherein the data processor comprises an expert system including machine learning capabilities operative to search the data in said second database and to identify the closest match having the highest probability of being correct based on said historical user verified closest matches.

15. The system of claim 13, further comprising a bi-directional communication device for enabling information to be sent to the system from a remote user and for enabling information to be obtained from the system by the remote user.

16. The system of claim 13, wherein said first database further includes storing one or more additional data fields for each medication in said first database, including data fields containing one or more of known dosages, routes of administration, and frequencies of administration, wherein said data processor is operative to compare said text data with the data in said data fields in said first database and to identify the one or more matched data fields that most closely matches the text data, and wherein said user interface displays to the user said one or more matched data fields for verification by the user.
